(19) United States
(12) Patent Application Publication
(10) Pub. No.: US 2024/0423513 A1
(43) Pub. Date: Dec. 26, 2024

(54) SUBCUTANEOUS GLUCOSE MONITORING SYSTEM

(71) Applicant: BIONIME CORPORATION, Taichung City (TW)

(72) Inventor: Chun-Mu HUANG, Taichung City (TW)

(73) Assignee: BIONIME CORPORATION, Taichung City (TW)

(21) Appl. No.: 18/750,405

(22) Filed: Jun. 21, 2024

Related U.S. Application Data
(60) Provisional application No. 63/509,622, filed on Jun. 22, 2023.

(51) Int. Cl. A61B 5/145; A61B 5/00

(52) U.S. Cl. CPC A61B 5/14532; A61B 5/0004; A61B 5/14503; A61B 5/7435

(57) ABSTRACT

A subcutaneous glucose monitoring system is disclosed. The subcutaneous glucose monitoring system includes a glucose measurement device that includes a sensor having a predetermined usage time length, and a reader comprising a processor, a wireless communication module, a memory, and a user interface configured to couple with the one or more processors and be operated by the user to set the pre-grace period and display the data. The processor is configured to execute the following steps according to the instruction: providing the pre-grace period for the user to be set on the user interface, wherein the pre-grace period is configured to be adjusted to a specific time length in response to a specific implant reaction; displaying none of the data on the user interface during the pre-grace period; enabling the user interface to display the data during a data display period in the predetermined usage time length after the pre-grace period expires; and when the data display period expires, causing the user interface to stop displaying the data.

### CROSS-REFERENCE TO RELATED APPLICATION AND CLAIM OF PRIORITY

This application claims the benefit of the U.S. Patent Application No. 63/509,622 filed on Jun. 22, 2023 at the United State Patent and Trademark Office, the disclosures of which are incorporated herein in their entirety by reference.

### FIELD OF THE INVENTION

The present invention is related to a subcutaneous glucose monitoring system, more particularly, the present invention is related to a subcutaneous glucose monitoring system that can cope with differences in the response of different living bodies to sensor implantation in order to prevent the display of inaccurate readings during the display period, and more specifically, the present invention is related to a subcutaneous glucose monitoring system that has a sufficient usage time length that meets the life time stipulated by the makers.

### BACKGROUND OF THE INVENTION

The continuous glucose monitoring system (CGM system) currently on the market for monitoring a patient's blood sugar/glucose concentration (level) includes a sensor and a transmitter. After the sensor is implanted, it measures the physiological signals, delivers the physiological signals to the transmitter, and gives a warning when one of the physiological signals is abnormal.

However, before the sensor can measure accurately, the sensor must be completely “wetting” or hydrated and in equilibrium with the glucose in the patient's body. Therefore, after the sensor is implanted subcutaneously, there must be a warm-up period before the sensor starts to measure, so that the patient/user can obtain an accurate reading of the glucose concentration/level in the patient's body.

The current CGM system usually requires a warm-up period of 30-120 minutes (i.e., 0.02-0.08 day) after the patient/user has activated it, and then it will enter the measurement period and display the reading(s) of the measured glucose level on the display of the CGM system.

In addition, after the sensor is implanted into the patient's body, it may cause wounds or even inflammatory responses to the patient's skin or subcutaneous tissues, thereby resulting in the signal measured by the sensor becoming unstable.

Usually, from the first day to the third day after implantation, which is called a start-up period, the accuracy of the data measured by the sensor is not good, so the mean absolute relative difference (MARD) of the curve of the data obtained during the start-up period is always greater than 10%, or even worse, greater than 19%-38%. Accordingly, after implantation, the patient must wait for a certain warm-up period to allow the sensor to equilibrate with the glucose in the patient's body, so that the continuous glucose sensor can stabilize and provide accurate readings.

As shown in “Table 18” in the previous document “PMA P150021: FDA Summary of Safety and Effectiveness Data, Abbott Diabetes Care, Inc., Sep. 23, 2016, pages 1-31”, which is shown in Table 1 below, the average relative difference between CGM-CM on the 1st day and even on the 2nd to 5th day is greater than 13%. At this time, the measurement of the CGM system is still extremely inaccurate. As written in the first paragraph of page 24 of the above-mentioned document, after wearing the CGM system, the measurement accuracy was extremely poor in the initial stage and gradually stabilized.

TABLE 1
Difference of daily CGM system to CM

| | Number of | Medium Absolute | Mean Absolute |
|---|---|---|---|
| | CGM-CM | Relative Difference | Relative |
| Day | pairs | (%) | Difference (%) |
| 1 | 2050 | 11.3 | 13.7 |
| 2-5 | 3973 | 10.9 | 13.2 |
| 6-9 | 2865 | 10.1 | 12.1 |
| 10-13 | 2175 | 8.4 | 10.2 |
| 14 | 1017 | 7.2 | 9.0 |

The service life of the CGM systems on the market is usually announced to be 3 days, 10 days, 14 days, or even 15 days. However, the service life announced by various makers, taking a service life of 15 days as an example, includes the warm-up period of 0.02 day and an unstable period of at least 1 to 5 days. The time length (or number of days) that the sensor can accurately measure are actually only at most 9.72-13.72 days. That is, the number of days that can be measured normally is less than the service life announced by the makers.

When the sensor is closer to the end of its service life, the accuracy of the sensor may also decline due to, for example, electrode passivation, enzyme degradation, delamination of the polymer film, biological contamination of the polymer film, cell fibrotic encapsulation, and circuit failure, and cause the signal measured by the biosensor in the latter period to be unstable. For details, please refer to the descriptions set forth in the Section “Introduction” on Pages 197-198 and FIG. 1 in the prior reference: “N. Wisniewski, M. Reichert, Methods for reducing biosensor membrane biofouling, Colloids and Surfaces B: Biointerfaces 18 (2000), pp 197˜219”.

Moreover, when the service life of the first sensor expires, the first sensor needs to be replaced by the second sensor. Another issue is how to maintain the physiological signals measured by the second sensor to be consistent with those measured by the first sensor, which needs to be improved for the conventional CGM system, so as to maintain continuous monitoring and timely alarm when the physiological signal is abnormal.

In view of this, the present invention provides a subcutaneous glucose monitoring system that can provide the user with a pre-grace period (or otherwise called an observation period) for coping with varying implant reactions to the implanted sensor in different living bodies. The data measured by the sensor is not played during the pre-grace period, but is displayed during the glucose data display period, and the time length of the glucose data display period is equal to the predetermined (or presumed) usage time length. Therefore, good accuracy can be maintained during the service life. Accordingly, the time length that the sensor can accurately measure the data equaling to the announced service life is truly achieved, so as to protect the users/consumers' rights and expectations to use the sensor with sufficient service life.

In addition, the present invention further provides a subcutaneous glucose monitoring system without a need to calibrate a second sensor when the second sensor is implanted to replace the first sensor before the first sensor is going to fail. Accordingly, the physiological signals/data/readings monitored by the system is uninterrupted, so timely alarm for an abnormal reading with accuracy can be continuously provided during the data display period transitioning between the first sensor and the second sensor. Therefore, the transition between the first and second sensors is simple.

Therefore, the Applicant has disclosed a subcutaneous glucose monitoring system to improve the problems of the prior art mentioned above.

### SUMMARY OF THE INVENTION

In accordance with one aspect of the present invention, a subcutaneous glucose monitoring system is disclosed. The subcutaneous glucose monitoring system comprises a glucose measurement device including a sensor having a predetermined/presumed usage time length, wherein at least a part of the sensor is configured to be implanted subcutaneously in a living body in contact with a biological fluid to output a measured signal or a data indicating a glucose level in the biological fluid, and a reader device configured to receive and display the data associated with the signal or convert the signal to the data, and provide a user with a pre-grace period for coping with varying implant reactions in different living bodies, wherein the user device comprises: one or more processors, a wireless communication module configured to couple with the one or more processors and wirelessly receive the signal or the data from the sensor, a memory configured to couple with the one or more processors and store an instruction, and a user interface configured to couple with the one or more processors and be operated by the user to set the pre-grace period and display the data, wherein the one or more processors are configured to execute the following steps according to the instruction: (a) providing an option unit for the user to set the pre-grace period on the user interface, wherein the pre-grace period is configured to be adjusted to a specific time length in response to a specific implant reaction; (b) displaying none of the data on the user interface during the pre-grace period; (c) enabling the user interface to display the data during a data display period in the predetermined usage time length after the pre-grace period expires; and (d) when the data display period expires, causing the user interface to stop displaying the data.

In accordance with another aspect of the present invention, a subcutaneous glucose monitoring system is disclosed. The subcutaneous glucose monitoring system comprises a glucose measurement device including a sensor having a predetermined/presumed usage time length, wherein at least a part of the sensor is configured to be implanted subcutaneously in a living body in contact with a biological fluid to output a measured signal or a data indicating a glucose level in the biological fluid, and a user device configured to receive and display the data associated with the signal or convert the signal to the data, and provide a user with a pre-grace period for coping with varying implant reactions with different living bodies, wherein the user device comprises: a processor, a wireless communication module configured to couple with the processor and wirelessly receive the signal or the data from the sensor, a memory configured to couple with the processor and store an instruction, and a user interface configured to couple with the processor and be operated by the user to set the pre-grace period and display the data, wherein the processor is configured to provide an option unit for the user to set the pre-grace period on the user interface, wherein the pre-grace period is configured to be adjusted to have a specific time length responding to a specific one of the varying implant reactions according to the instruction.

In accordance with another aspect of the present invention, a subcutaneous glucose monitoring system is disclosed. The subcutaneous glucose monitoring system comprises a glucose measurement device including a sensor having a predetermined/presumed usage time length by a factory end, wherein at least a part of the sensor is configured to be implanted subcutaneously in a living body in contact with a biological fluid to output a measured signal or a data indicating a glucose level in the biological fluid, and a reader device configured to receive and display the data associated with the signal or convert the signal to the data, wherein the user device comprises: a processor, a wireless communication module configured to couple with the processor and wirelessly receive the signal or the data from the sensor, a memory configured to couple with the processor and store an instruction, and a user interface configured to couple with the processor and be operated by the user to set the pre-grace period and display the data, wherein the processor is configured to provide a user with a pre-grace period for coping with varying implant reactions in different living bodies and enable the user interface to display the data during a data display period in the predetermined/presumed usage time length after the pre-grace period expires according to the instruction.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a schematic diagram of a composition of a subcutaneous glucose monitoring system for monitoring a patient's glucose concentration according to one embodiment of the present invention;

FIG. 2 is a schematic diagram of working principle of the subcutaneous glucose monitoring system for monitoring a patient's glucose concentration according to the present invention;

FIG. 3 is a schematic diagram of a reader device in the subcutaneous glucose monitoring system according to one embodiment of the present invention;

FIGS. 4A and 4B are steps executed by a processor of the reader device in the subcutaneous glucose monitoring system according to the present invention;

FIGS. 5A, 5B, 6A, 6B, 6C, 7A and 7B are images displayed on the screen of the user interface in the subcutaneous glucose monitoring system according to one embodiment of the present invention;

FIG. 8 is a schematic diagram showing a patient implanted with a second sensor according to one embodiment of the present invention;

FIG. 9 is a schematic diagram showing time lines of a warm-up period, a pre-grace period and a display period of a sensor according to one embodiment of the present invention;

FIG. 10 is a schematic diagram showing a combination of the time lines of a first sensor and a second sensor according to one embodiment of the present invention;

FIG. 11 is a schematic diagram showing a combination of the time lines of a first sensor and a second sensor according to another embodiment of the present invention;

FIG. 12 is a flow diagram showing the steps executed by the processor according to one embodiment of the present invention;

FIG. 13 is a flow diagram showing the steps executed by the processor according to another embodiment of the present invention; and

FIG. 14 is a schematic graph showing a curve of continuous data measured by the subcutaneous continuous glucose monitoring (CGM) system according to the present invention and the data measured by a convention glucose monitoring (BGM) system.

### DETAILED DESCRIPTION OF THE PREFERRED EMBODIMENTS

The invention proposed in the present application will be fully understood by the following embodiments, so that the person skilled in the art can implement it. However, the implementation of the present application is not limited by the following embodiments. The person skilled in the art can still deduce other embodiments based on the spirit of the embodiments disclosed herein, but these embodiments should all fall within the scope of the present invention.

Please refer to all figures of the present invention when reading the following detailed description, wherein all figures of the present invention demonstrate different embodiments of the present invention by showing examples, and help the skilled person in the art to understand how to implement the present invention. The present examples provide sufficient embodiments to demonstrate the concept of the present invention, each embodiment does not conflict with the others, and new embodiments can be implemented through an arbitrary combination thereof; that is, the present invention is not restricted to the embodiments disclosed in the present specification.

Unless there are other restrictions defined in the specific example, the following definitions apply to the terms used throughout the specification.

In one embodiment of the present invention, the service life of the sensor can reach 18 to 21 days. As mentioned above, if a commercially available sensor has a product life of 15 days, In the case where the warm-up period plus the data stabilization period is 1 day, the data measured during the period from the 2nd day to the 15th day can actually be presented, i.e., there is a data display period of 14 days in total. In contrast, the method for displaying the data according to the present invention is to display the data measured from at least the 2nd day to the 16th day, i.e., there is a data display period of at least 15 days in total by setting a pre-grace period, such as 1 day, after the sensor is implanted, so as to provide the patient/user to enjoy the data display period having high accuracy, provide accurate data during the data display period, and allow the product to meet the claimed usage time length to protect the patient/user's rights. Even more, since the service life of the sensor of the present invention can reach 18 to 21 days, it means that the sensor (hereinafter called the first sensor) of the present invention can still operate normally after the 16th day. The extra period from the 16th day to the 18th day (three days in total) or from the 16th to the 21th day (six days in total) can serve as a post-grace period, for continued measurements during the pre-grace period of a next sensor (hereinafter called the second sensor) subsequently implanted, or to extend the adjustable pre-grace period range.

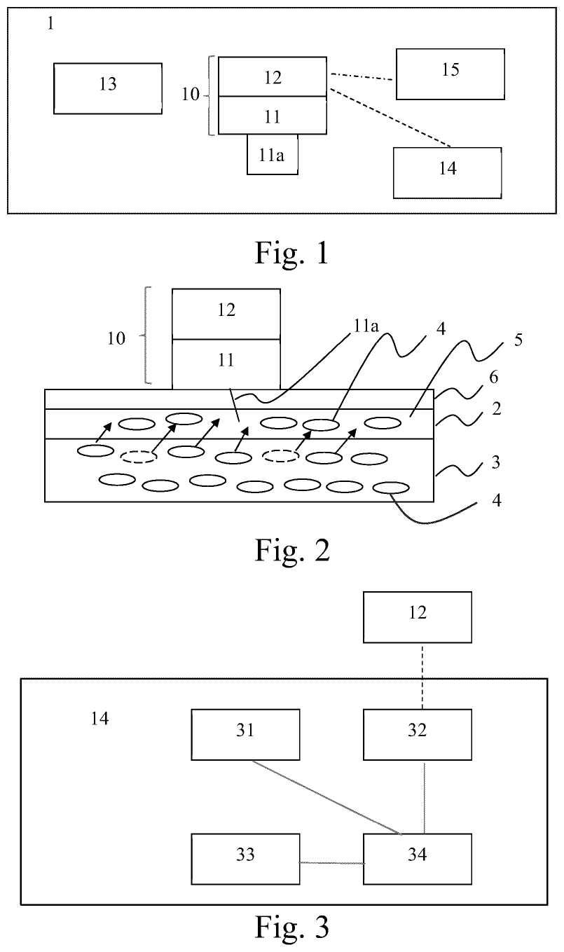

FIG. 1 is a schematic diagram of a composition of a subcutaneous glucose monitoring system for monitoring a patient's glucose concentration according to one embodiment of the present invention. FIG. 2 is a schematic diagram of working principle of a subcutaneous glucose monitoring system for monitoring a patient's glucose concentration according to one embodiment of the present invention. As shown in FIG. 1, the subcutaneous glucose monitoring system 1 of the present invention includes a glucose measurement device 10, which includes a sensor module 11 (including a sensor 11a implanted subcutaneously), an transmitter 12 for transmitting physiological signals (or simply called signals) or physiological data (or simply called data) measured by the sensor 11a and for connecting to the sensor module 11 after at least a part of the sensor 11a is implanted, an inserter 13 for subcutaneously implanting the sensor 11a, a reader device 14 (which can be a mobile phone or any receiver with relevant APP installed therein, for example) for receiving signals/data transmitted from the transmitter 12, and a charger 15 for charging the transmitter 12. The frequency at which the sensor 11a measures physiological signals can be, for example, every minute, every 2 minutes, every 5 minutes, every 10 minutes, every 15 minutes, every 30 minutes, every hour, etc. As shown in FIG. 2, at least a part of the sensor 11a is implanted into the living body through the inserter 13 to penetrate through the skin 6 and contact the biological fluid 5 (for example, the tissue fluid that penetrates from the blood vessel 3 into the subcutaneous tissue 2), and applying a voltage through the transmitter 12 to the electrodes (not shown) on the sensor 11a for detecting the electrochemical reaction occurring between the electrodes and the glucose 4 in the biological fluid 5, so as to measure the physiological signal representing the level of the glucose 4. Then the transmitter 12 transmits the physiological signal measured by the sensor 11a, or further converts the physiological signal into physiological data (according to the function design of the transmitter and the receiver for signal conversion and transmission), and transmits the physiological signal/physiological data to the reader device 14. The reader device 14 is used for receiving the physiological data transmitted from the transmitter 12, or converting physiological signals into physiological data, and further displaying the physiological data on the user interface of the reader device 14. In another embodiment, the sensor 11a can be implanted subcutaneously in combination with a micro-transmitting device.

FIG. 3 is a schematic diagram of a reader device in the subcutaneous glucose monitoring system according to one embodiment of the present invention. As shown in FIG. 3, the reader device 14 of the present invention includes a user interface 31, a communication module 32 (which can be wired or wireless), a memory 33 and a processor 34. The user interface 31 is, for example, a mobile phone or other handheld human-machine interface. The communication module 32 is configured to connect with the transmitter 12 in the glucose measuring device 10 through a wired communication or a wireless communication (such as Bluetooth® or WIFI) and obtain the physiological signal (or simply signal) or physiological data (or simply data). The processor 34 is connected to the user interface 31, the memory 33 and the communication module 32 respectively. The processor 34 in the reader device 14 converts the physiological signals obtained from the communication module 32 into physiological data, stores the data to the memory 33, and displays the processed or unprocessed physiological data on the user interface 31. The signal measured by the sensor 11a is a glucose measurement signal, and the data is a glucose reading converted from the glucose measurement signal.

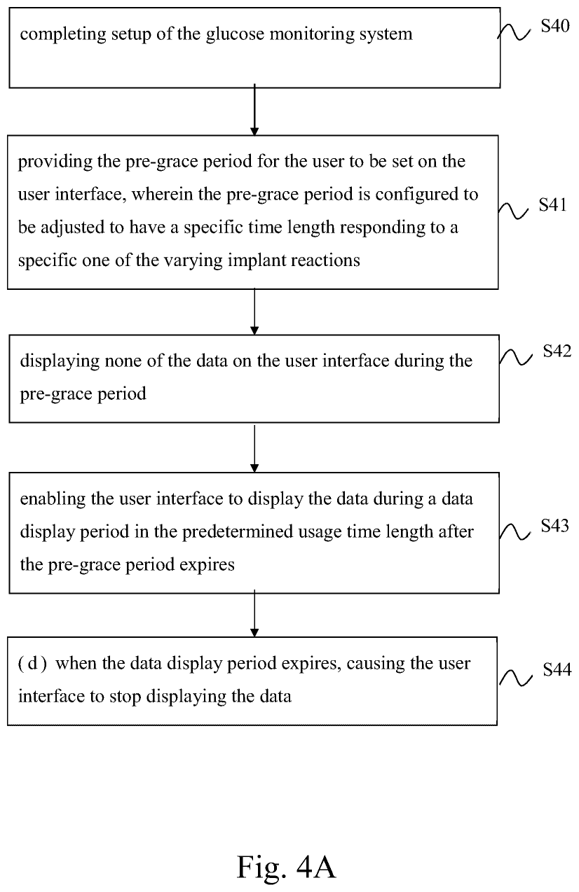

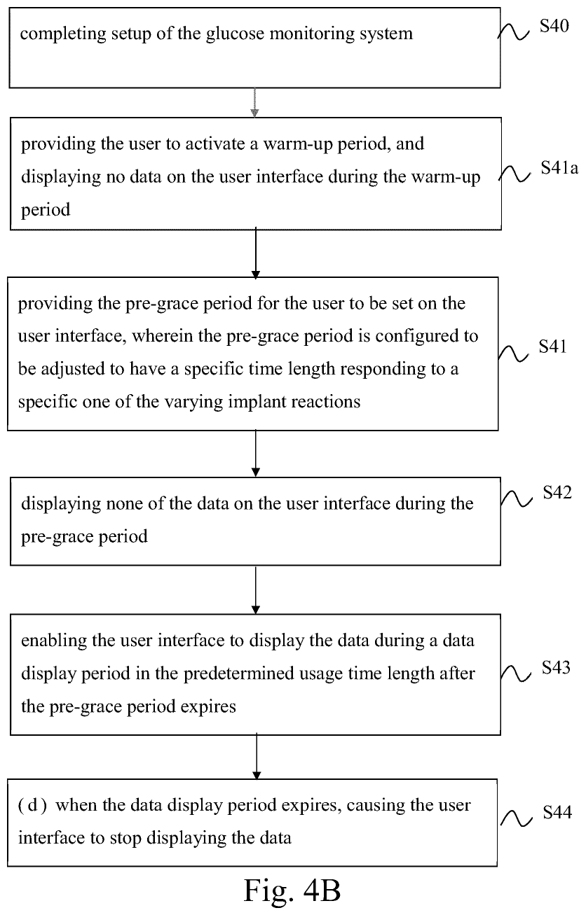

FIG. 4A and FIG. 4B are steps executed by a processor of the reader device in the subcutaneous glucose monitoring system according to the present invention. Please refer to FIG. 1 to FIG. 3, a subcutaneous glucose monitoring system 1 according to an embodiment of the present invention includes a glucose measuring device 10 and a reader device 14. The glucose measuring device 10 includes a sensor module 11 with a predetermined (or presumed) usage time length defined at the factory end, and at least a part of the sensor 11a of the sensor module 11 is implanted subcutaneously in the living body to contact with the biological fluid 5 for measuring a signal indicating the level of glucose 4 in the biological fluid 5. The reader device 14 is used to pair with the transmitter 12 of the glucose measuring device 10. When the glucose monitoring system 1 is completely set up, the reader device 14 is configured to receive the signal and convert the signal to data, or receive the data, and then display the data. The reader device 14 is further configured to provide the user to set a pre-grace period (or an observation period) responding to implant reaction differences from different living bodies. The reader device 14 includes one or more processors 34, a wireless communication module 32, a memory 33, and a user interface 31. The wireless communication module 32 is configured to couple with the one or more processors 34 and wirelessly receive the signal or data from the sensor module 11. The memory 33 is configured to couple with the one or more processors 34 and to store instructions. The user interface 31 is configured to couple with the one or more processors 34 and for the user to set the pre-grace period (or called observation period) and display the data, wherein the one or more processors 34 are configured to execute the steps as shown in FIG. 4A according to the instructions including: completing setup of the glucose measurement device 10 (S40); providing the pre-grace period for the user to be set on the user interface 31, wherein the pre-grace period is configured to be adjusted to have a specific time length responding to a specific one of the varying implant reactions (S41); displaying none of the data on the user interface 31 during the pre-grace period (S42); enabling the user interface 31 to display the data during a data display period in the predetermined/presumed usage time length after the pre-grace period expires (S43); and when the data display period expires, causing the user interface 31 to stop displaying the data (S44). In addition, as shown in FIG. 4B, in another embodiment, before Step (S41), the following steps may also be included: providing the user to activate a warm-up period, and displaying no data on the user interface 31 during the warm-up period (S41a). The warm-up period is usually a fixed value that is built in the system. In another embodiment, the warm-up period is user adjustable.

The time length adjustable for the warm-up period is within the range of no more than 1 day, no more than 2 days, no more than 3 days, or no more than 5 days. Preferably, the adjustable time length is at least 20 minutes. Furthermore, the setting of the adjustable time length of the pre-grace period is determined based on one or a combination selected from a group consisting of an implant reaction caused by the living body to the sensor, a lifetime of the sensor, the predetermined usage time length, a function of at least one system parameter of the sensor. The system parameters of the sensor are determined at least based on a sensitivity of the sensor.

The range of setting the adjustable time length of the pre-grace period can be defined at the factory end.

The upper limit and the low limit of the range for setting the time length of the pre-grace period can be defined based on the service life of the sensor, a standard usage time length of the sensor, and a function of the system parameters of the sensor, and the adjustable time length is within a range from half a day to one day.

The user interface is at least configured to display an option unit for the user to reject, or accept to set the adjustable time length. Furthermore, the adjustable time length may be set from one of a plurality of preset fixed time lengths or from a time length defined by the user.

The implant reaction differences from different living bodies for the sensor implantation include the differences in changes in physiological environment, passivation of sensor electrodes, or cell fibrotic encapsulation of the sensor resulting from biological and immunological responses between the body of the patient and at least a part of sensor.

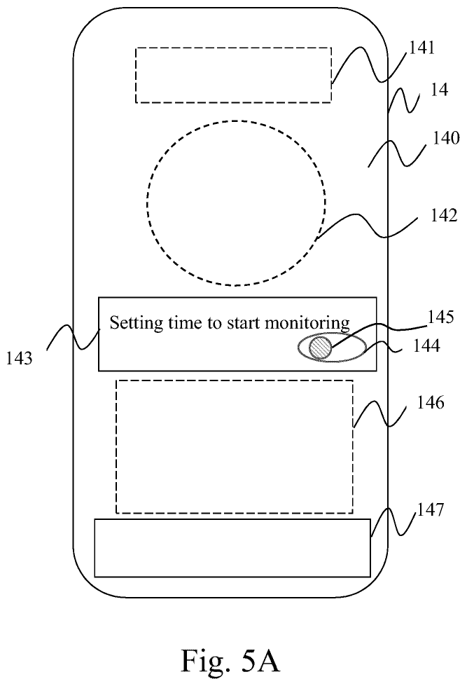

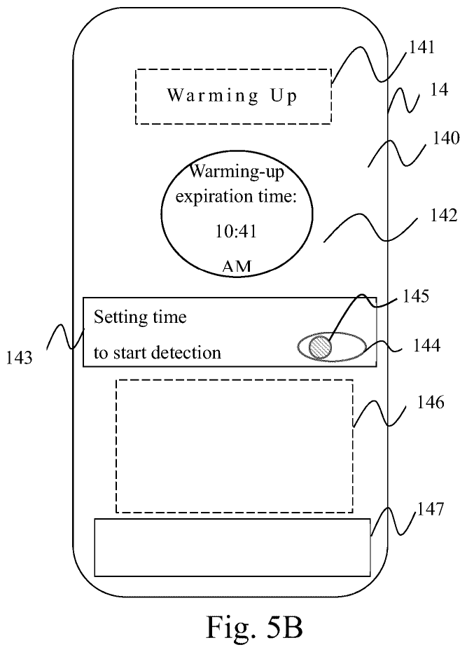

FIGS. 5A, 5B, 6A, 6B, 6C, 7A and 7B are images displayed on the screen of the user interface in the subcutaneous glucose monitoring system according to one embodiment of the present invention. The embodiment described below is applicable for a mode in which, after at least a part of the sensor 11a is implanted, a warm-up period is activated first, and then a decision regarding whether a pre-grace period is to be activated has to be made. However, because there are a lot of designs and arrangements for the screen of the user interface to set the warm-up period and the pre-grace period for the sensor, the images shown in FIG. 5A to FIG. 7B are the embodiments of the present application that make them simple and clear to the reader, but the embodiments of the present invention are not limited thereto.

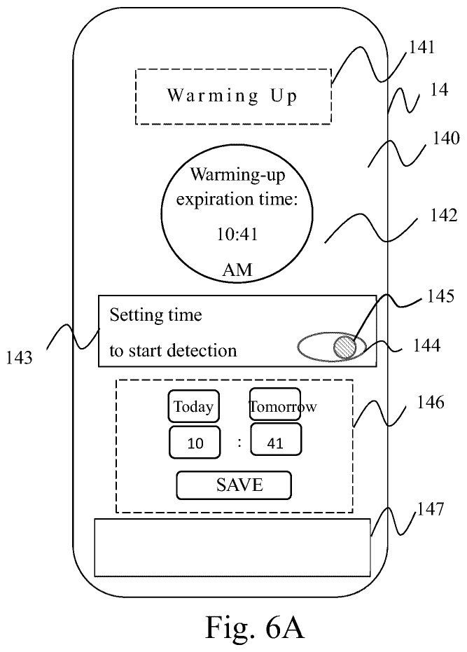

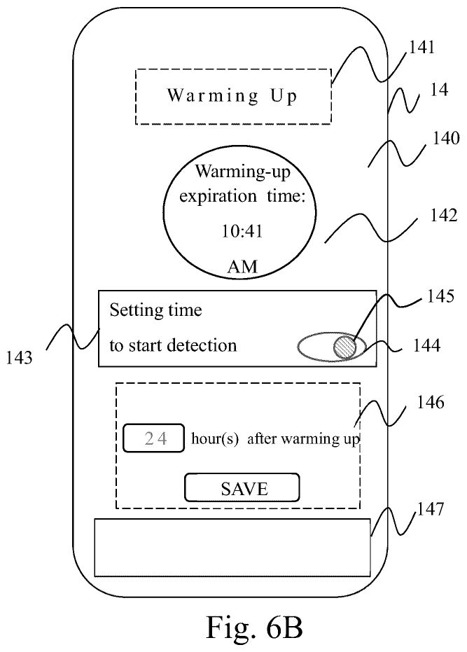

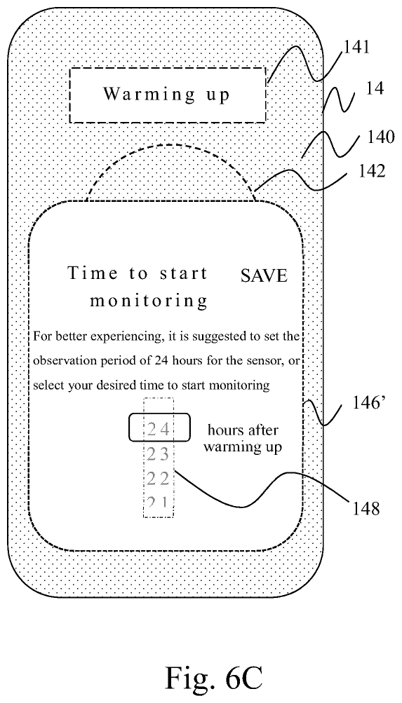

As shown in FIG. 5B, after the user interface 31 is turned on and the related application program (App) is initiated, the warm-up period of the sensor 11a starts, and the phrase “warming up” appears in the display box 141 in the display area 140 of the user interface 14, and the phrase “warming-up expiration time” and a scheduled completion time of the warm-up, for example, “10:41 AM” appear in the display box 142. Meanwhile, the phrase “setting time to start detection” and a switch icon 144 for the user to select appear in the option unit 143. When the selection button 145 is kept on the left side of the switch icon 144, it indicates that the user does not intend to set the time to start monitoring, meaning that the user chooses to reject the setting and set the pre-grace period to 0; if the selection button 145 is moved to the right side of the switch icon 144, as shown in FIG. 6A, it indicates that the user intends to set the time to start monitoring, meaning that the user chooses to set the pre-grace period time. At this moment, the display box 146 displays a time selectable for the user to set the time to start monitoring, for example, 10:41 tomorrow, and then press “Save” button in the display box 146 to complete the saving and setting the time to start monitoring. In this case, the time to start monitoring is 10:41 the next day, which means that the pre-grace period is set as 24 hours. In addition, during the warm-up period (i.e., in the aforementioned step S41), the user interface does not display the data during the warm-up period when the warm-up period is activated by the one or more processors.

Alternatively, when the selection button 145 is moved to the right side of the switch icon 144, as shown in FIG. 6B, the display box 146 will display the time going to start monitoring, such as a time point after a period from the warm-up period, and then press the “Save” button in the display box 146 to complete the saving and setting the time to start monitoring. Assuming that the time to start monitoring is set to be 24 hours after the warm-up period ends/expires, the sensor will have a 24-hour pre-grace period, and the time to start measurement will be 10:41 the next day.

In other words, if in the step S41 the user chooses to accept, then the step S41 also includes the following steps: causing the user interface to provide the user to confirm the pre-grace period set by the user; and causing the user interface to display the required time lapse or the expiration time message to reach the time to start monitoring. The format displayed for the required time lapse or the expiration time message can be an expiration time point, a countdown timing, a positive timing, or a combination thereof.

As shown in FIG. 5B, FIG. 6A and FIG. 6B, the display box 147 that includes one or more function selection buttons, e.g. HOME, LOG, REPORT, MORE, NEXT PAGE, PREVIOUS PAGE, etc., are all adapted to the design of the App for the users to operate the user interface, and the details of which are not necessary to explain here.

As shown in FIG. 6C, the user intends to set the time to start monitoring, meaning that the user chooses to set the pre-grace period. At this moment, the option unit 148 will appear in the display box 146. The option unit 148 has an upright sliding selection bar, alternatively, a horizontal sliding selection bar is also available. The user can set the time by sliding the selection bar that displays the number of hours to start measurement after the warm-up period ends, for example, 24 hours after the warm-up period, and then the user can press the “Save” button in the display box 146′ to complete the saving and setting the time to start monitoring. As a result, the pre-grace period is set to be 24 hours. In addition, during the warm-up period (i.e., in the aforementioned step S41), the user interface does not display the data during the warm-up period when the warm-up period is activated by the one or more processors.

In addition, as shown in FIG. 4A, in another embodiment, the step S40 of providing the pre-grace period for the user to be set on the user interface wherein the pre-grace period is configured to be adjusted to have a specific time length responding to a specific one of the varying implant reactions is omitted, and the image shown in the screen of the user interface looks like the one as shown in FIG. 5A. The other image on the screen for setting the pre-grace period can be implemented by referring to FIG. 6A to FIG. 6C. In another embodiment, the step S40 of providing the pre-grace period for the user to be set on the user interface wherein the pre-grace period is configured to be adjusted to have a specific time length responding to a specific one of the varying implant reactions may be executed after the step S41.

When the service life of the sensor 11a (hereinafter referred to as the first sensor 11a) is going to expire, it is necessary to implant a second sensor 11a′ into the patient's body (or a living body) 500 for continuously monitoring the glucose level. The method for the pairing and connection between each sensor and the user interface 14 is not necessary to discuss here.

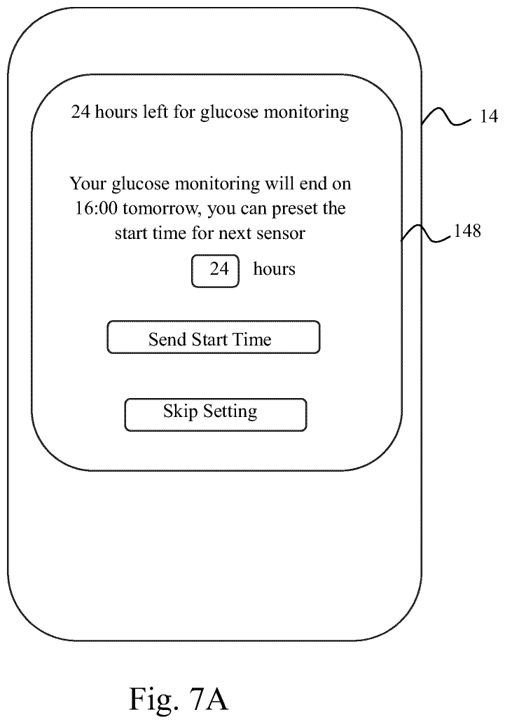

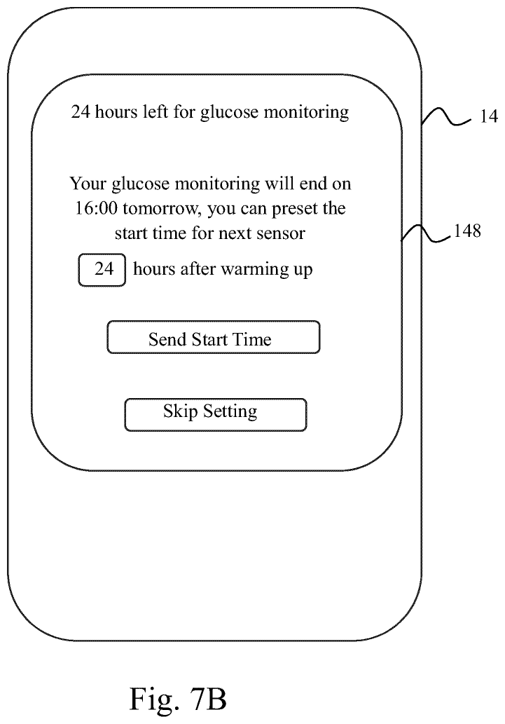

In order not to interrupt the glucose monitoring and warning to a patient (or a living body) 500, for example, 24 hours before the time that the first sensor 11a is about to expire (for example, to expire at 16:10 tomorrow), as what are shown in FIG. 4A and FIG. 7A, the option unit 148 appears in the user interface 14, and a prompt appearing in the option unit 148 includes the end time for monitoring of the first sensor 11a, a “Send Start Time” button, and a “Skip Setting” button. If the user intends to set the second pre-grace period of the second sensor 11a′, for example, after 24 hours, to start monitoring by the second sensor 11a′, and then press the “Send Start Time” button in the option unit 148 button to complete the saving and setting the time to start monitoring. As what are shown in FIG. 4B and FIG. 7B, the option unit 148 appears in the user interface 14, and a prompt appearing in the option unit 148 includes the end time for monitoring of the first sensor 11a, a “Send Start Time” button, and a “Skip Setting” button, so that the time length of the second per-grace period of the second sensor 11a′ can be preset. If the user intends to set the second pre-grace period of the second sensor 11a′ starting at a time point, say, 24 hours after the second warm-up period of the second sensor 11a′ expires to start monitoring by the second sensor 11a′, and then press the “Send Start Time” button in the option unit 148 button to complete the saving and setting the time to start monitoring, then the second sensor 11a′ has a second pre-grace period of 24 hours, and the time to start measurement will be 16:10 the next day. If the user does not intend to set the second pre-grace period, he can press the “Skip Setting” button in the option unit 148 to cause the second sensor to skip the second pre-grace period and start measurement directly when the warm-up period ends. The method for setting the pre-grace period shown in FIG. 7A or FIG. 7B is only an example for illustration, and other modified embodiments can be made with reference to the above descriptions, and thus will not be described again here.

Therefore, the reader device of the present invention enables the user interface to remind the user to prepare for installation of the second sensor at the end or close to an end of the data display period of the first sensor in step S43 executed by the processor. After the installation of the second sensor is completed, a second pre-grace period that can be set by the user is provided.

In addition, at the end or close to the end of the data display period in the aforementioned step S43, the reader device enables the user interface to display an option unit (not shown) for setting a post-grace period of the first sensor. Although not shown in the figure, the option unit can also be realized by a person skilled in the art with reference to the option unit previously mentioned. When the option unit for setting the post-grace period is selected to be accepted by the user, the data will continue to be displayed for a period of time when the first data display period expires. The post-grace period may be set to be, for example, 24 hours or less or 12 hours or less.

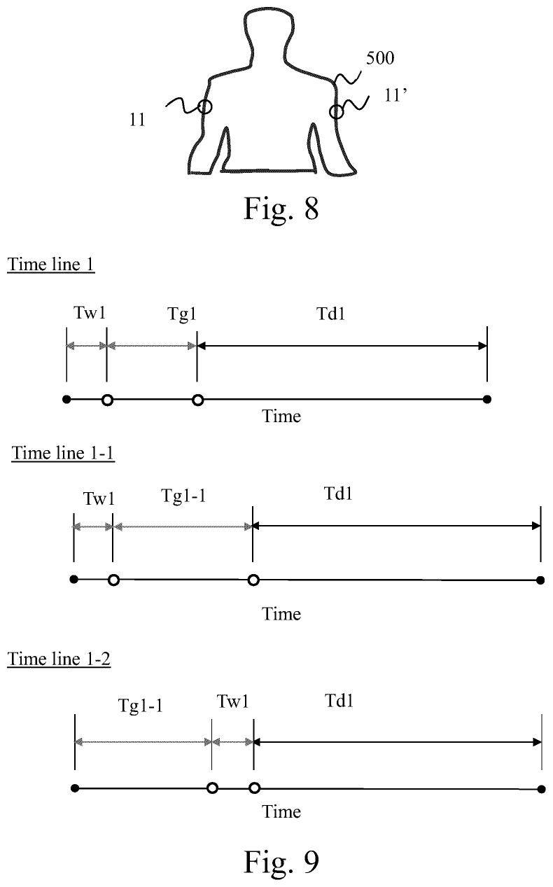

FIG. 8 is a schematic diagram showing a patient implanted with a second sensor according to one embodiment of the present invention. As shown in FIG. 8, the patient 500 has the first sensor installed on one arm. Before the expiration of the first sensor 11a, the user interface reminds the user to prepare for installation of the second sensor 11a′, for example, to be installed on the patient's other arm. In this way, before the second sensor 11a′ can accurately display data, the first sensor 11a can still display data, so that the glucose monitoring on the patient 500 is not interrupted.

FIG. 9 is a schematic diagram showing time lines of a warm-up period, a pre-grace period and a display period of a sensor according to one embodiment of the present invention. As shown in FIG. 9, the time line 1 includes a first warm-up period (Tw1), a preset first pre-grace period (Tg1), and a first display period (Td1). The time line 1-1 and time line 1-2 are variations of the time line 1. The time line 1-1 differs from the time line 1 in that its pre-grace period is set by the user. The time line 1-2 differs from the time line 1 in that the first pre-grace period (Tg1-1) is executed first and the warm-up period (Tw1) is executed later.

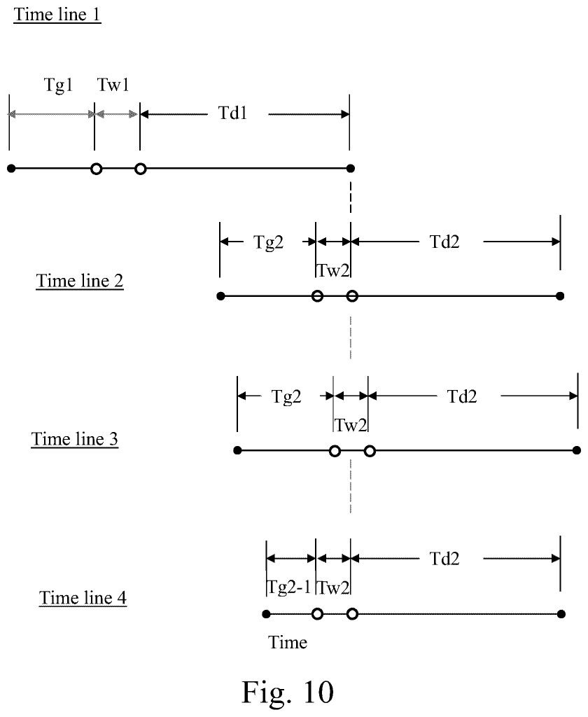

FIG. 10 is a schematic diagram showing a combination of the time lines of a first sensor and a second sensor according to one embodiment of the present invention. As shown in FIG. 10, the time line 1 includes the first warm-up period (Tw1) of the first sensor, the preset first pre-grace period (Tg1) and the first display period (Td1), the time line 2 to the time line 4 are variations for the second sensor and include a preset second pre-grace period (Tg2) or a user-defined second pre-grace period (Tg2-1), a second warm-up period (Tw2) of the second sensor, and a second data display period (Td2). The time line 2 includes a second pre-grace period (Tg2) set by the user, a second warm-up period (Tw2) and a second data display period (Td2), wherein the time of the second warm-up period (Tw2) ends at the same time as the first data display period (Td1) ends, and thus the patient's glucose can be monitored without interruption. The time line 3 differs from the time line 2 in that the end time of the second warm-up period (Tw2) of the time line 3 is later than the end time of the first display period (Td1) of the first sensor. This may be because the user installs the second sensor too late, resulting in the monitoring being interrupted. The time line 4 differs from the time line 2 in that the second pre-grace period (Tg2-1) in the time line 4 set by the user is shorter than the second pre-grace period (Tg2) in the time line 2, while the second warm-up period in them ends at the same time as the first data display period (Td1) of the first sensor ends, and thus the patient's glucose can be monitored without interruption. Of course, the sequence of the second pre-grace period and the second warm-up period of the second sensor can be interchanged without further explanation.

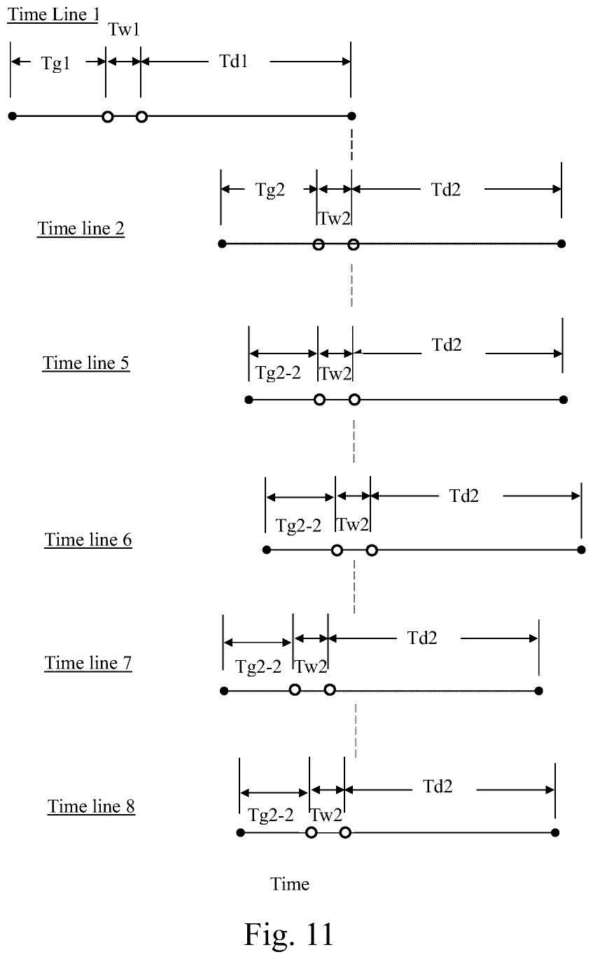

FIG. 11 is a schematic diagram showing a combination of the time lines of a first sensor and a second sensor according to another embodiment of the present invention. As shown in FIG. 11, the time line 1 includes the first warm-up period (Tw1), the preset first pre-grace period (Tg1) and the first display period (Td1) of the first sensor, the time line 2 and the time lines 5 to 8 are variations for the second sensor, and include a preset second pre-grace period (Tg2) or a user-defined second pre-grace period (Tg2-2), a second warm-up period (Tw2) and the data display period (Td2) of the second sensor. The time line 2 includes a user-defined second pre-grace period (Tg2), a second warm-up period (Tw2) and a second data display period (Td2), wherein the end time of the second warm-up period (Tw2) is the same as the end time of the first display period (Td1) of the first sensor, and thus the patient's glucose can be monitored without interruption. The time line 5 differs from the time line 2 in that the second pre-grace period (Tg2-2) of the time line 5 set by the user is shorter than the second pre-grace period (Tg2) of the time line 2, while the second warm-up period (Tw2) in them ends at the same time as the first data display period (Td1) of the first sensor ends, and thus the patient's glucose can be monitored without interruption. The time line 6 differs from the time line 2 in that the end time of the second warm-up period (Tw2) is later than the end time of the first display period (Td1) of the first sensor. This may be because the user installs the second sensor too late, resulting in monitoring interruptions. The time line 7 differs from the time line 2 in that the end time of the second warm-up period (Tw2) is earlier than the end time of the first display period (Td1) of the first sensor. The time line 7 differs from the time line 2 in that the end time of the second warm-up period (Tw2) of the time line 7 is earlier than the end time of the first display period (Td1) of the first sensor, and slighter earlier than the second warm-up period (Tw2) of the time line 2. Of course, the sequence of the second pre-grace period and the second warm-up period of the second sensor can be interchanged without further explanation.

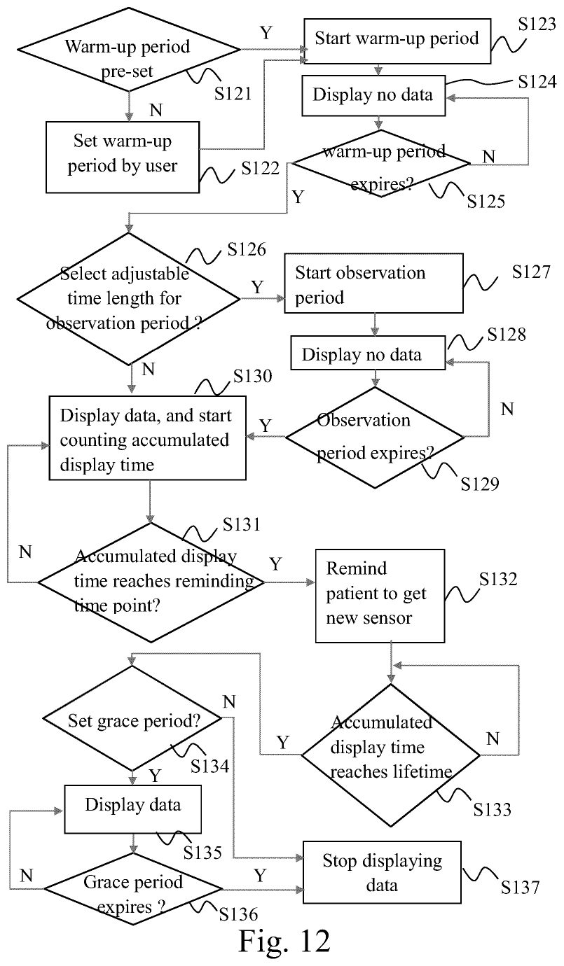

FIG. 12 is a flow diagram showing the steps executed by the processor according to one embodiment of the present invention, wherein if the sensor has both a warm-up period and a pre-grace period, the warm-up period is before the pre-grace period. As shown in FIG. 12, first, it is determined whether each installed sensor (no matter which sensor is installed) has a preset warm-up period (S121): if not, the user sets the length of the warm-up period (S122), and then activates the warm-up period (S123); if yes, the warm-up period is activated (S123). After the warm-up period is activated, the user interface does not display data (S124) until the warm-up period expires (S125).

The next step is to set the pre-grace period. First, it is determined whether the user chooses to set the adjustable length of the pre-grace period (S126): if no, the data is displayed on the user interface (S130); if yes, the pre-grace period is activated (S127), during which the user interface does not display the data (S128) until the pre-grace period expires (S129) and displays the data (S130).

According to another embodiment of the present invention, during the process of displaying data on the user interface (S130), at the same time, optionally, the accumulation of the time that the data has been displayed is counting (S130) for the purpose that, when a specific accumulated display time is reached (i.e., when time to remind the user has been reached), the user is reminded to prepare for installation of the second sensor. Therefore, it is determined whether the accumulated display time reaches the time for reminding the user (S131): if not, keep displaying the data and keep counting the accumulated display time (S130); if yes, the user is reminded to prepare a new sensor (S132), and continue to count the accumulated display time, and then it is determined whether the service life of the sensor ends/expires (S133): if not, continue to display the accumulated display time; if yes, it is allowed for the user to choose whether to extend the display time (S134): if not, stop displaying the data (S137); if yes, keep displaying the data (S135), and it is determined whether the extended display time has expired (i.e., the extended display time has been reached) (S136): if not, keep displaying the data (S135); if yes, stop displaying the data (S137).

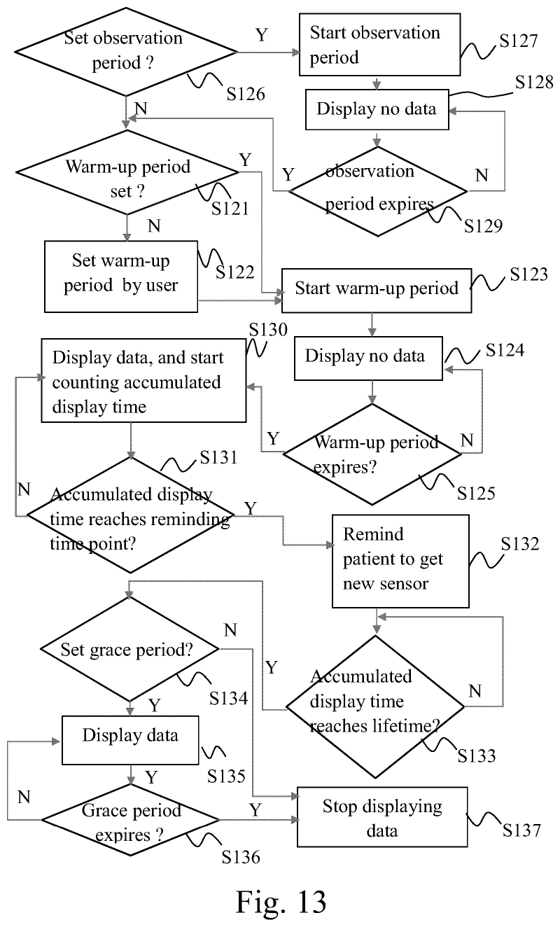

FIG. 13 is a flow diagram showing the steps executed by the processor according to another embodiment of the present invention, wherein if the sensor has both a fixed warm-up period and a pre-grace period, the warm-up period will be after the pre-grace period. As shown in FIG. 13, first, it is determined by the user whether to set the length of the pre-grace period for each installed sensor (no matter which sensor is installed) (S126): if not, it is determined whether the length of the warm-up period has been set (S12), and then the warm-up period is activated (S123); if yes, the warm-up period is activated directly (S123). After the warm-up period is activated, the user interface does not display data (S124) until the warm-up period ends/expires (S125), and then the data is displayed on the user interface (S130).

Next, similarly, according to another embodiment of the present invention, during the process of displaying data on the user interface (S130), at the same time, optionally, the accumulation of the time that the data has been displayed is being counted (S130) for the purpose that, when a specific accumulated display time is reached (i.e., when time to remind the user has been reached), the user is reminded to prepare for installation of the second sensor. Therefore, it is determined whether the accumulated display time reaches the time for reminding the user (S131): if not, keep displaying the data and keep counting the accumulated display time (S130); if yes, the user is reminded to prepare a new sensor (S132), and continue to count the accumulated display time, and then it is determined whether the service life of the sensor ends/expires (S133): if not, continue to display the accumulated display time; if yes, it is allowed for the user to choose whether to extend the display time (S134): if not, stop displaying the data (S137); if yes, keep displaying the data (S135), and it is determined whether the extended display time has expired (i.e., the extended display time has been reached) (S136): if not, keep displaying the data (S135); if yes, stop displaying the data (S137).

In addition, if the mean absolute relative difference (MARD) is used to evaluate the difference between the CGM system reading and the reference measurement value, it can serve as an indicator of data accuracy. According to the present invention, during the glucose data display period (i.e., the measurement period), the MARD value of the data (or the data set collected from the data) obtained from the sensor during its service life (or called survival period) is less than or equal to 10%, preferably less than or equal to 9%, and more preferably less than or equal to 8%. The survival period of the sensor refers to the time during which the glucose measurement device 10 can operate normally during the glucose data display period when the sensor is used, that is, the time period during which the sensor adheres to the skin and functions effectively.

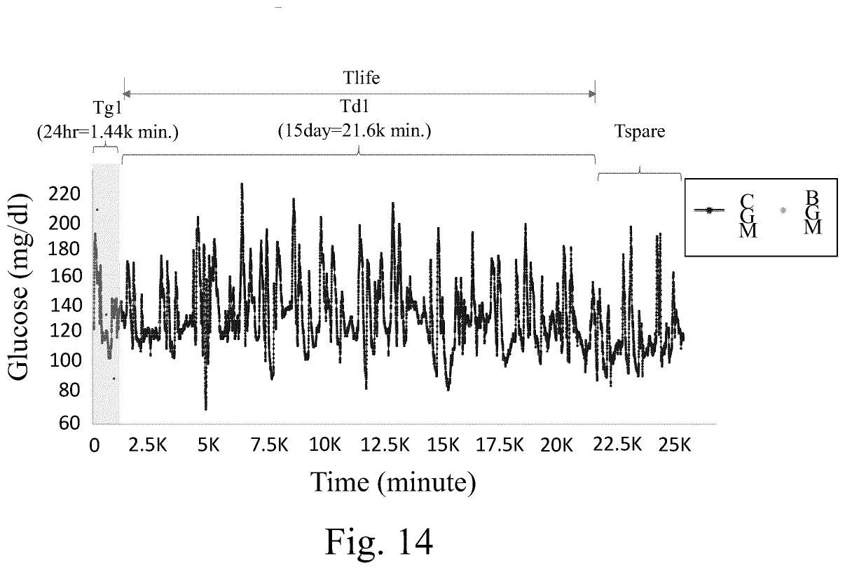

FIG. 14 is a schematic graph showing a curve of continuous data measured by the subcutaneous continuous glucose monitoring (CGM) system according to the present invention and the data measured by a convention glucose monitoring (BGM) system. As shown in FIG. 14, the horizontal axis represents time and the longitudinal axis represents glucose concentration. The curve constituted from the data that are measured by the subcutaneous glucose monitoring system of the present invention shows continuous and regular monitoring of the patient's glucose concentration. The subcutaneous glucose monitoring system also provides alarm when the data is abnormal. As to the convention glucose monitoring (BGM) system, the patient or the carer can only rely on the data measured from the blood sample taken each time. During the period between the two samplings, it is impossible to know in time if the glucose level goes abnormal. Taking the first sensor as an example, during the service life (Tlife) of the first sensor, at least 95% of the data will be within 25% of the glucose reference value. As shown in FIG. 14, the data shown during the glucose data display period maintain better accuracy. In addition, the period on the left side of the displayed data is the pre-grace period Tg1 preset as 24 hours (i.e., 1.44 thousand minutes) for example, the period in the middle is the data display period Td1 being 15 days (i.e., 21.6 thousand minutes) for example, and the spare period on the right side Tspare can be used to include the post-grace period. In practice, the post-grace period will not occupy the entire reservation period Tspare. In this way, the pre-grace period Tg1 can be set by the user to be greater than 24 hours, which means that the user can have greater flexibility for setting the pre-grace period Tg1.

According to the glucose monitoring system according to an embodiment of the present invention, the processor does not enable the user device to display the data indicating the glucose level during the pre-grace period, and the pre-grace period can be preset as 1 day.

After at least part of the sensor of the present invention is implanted subcutaneously, a warm-up period can be set. The warm-up period depends on the individual patient's physical condition and can be preset at the factory end or set by the user from the range from 1 hour to 3 days for example, and the measured data/readings are not displayed during the warm-up period.

The measured data can be pre-calibrated at the factory end or calibrated by a reference instrument, and then displayed. When the warm-up period ends or the pre-grace period ends (whichever comes later), the measurement period/data display period starts and the data is displayed, and the number of days using the sensor starts to be calculated until the preset usage day expires/ends.

According to an embodiment of the present invention, the state of the sensor being in either the warm-up period or the pre-grace period can be shown on the user interface without displaying the data. In addition, the actual number of days that the subcutaneous glucose monitoring system can provide valid measurement and display data is consistent with the announced number of days of use of the product.

According to an embodiment of the present invention, the data used to calibrate the second sensor are not the data obtained from the first sensor.

According to an embodiment of the present invention, the glucose measuring device 10 applies a pulse voltage to the sensor 11a during the pre-grace period to accelerate the sensor 11a to reach its optimum status to ensure that the user can enjoy the best performance of outputting the data during use of the sensor/system.

According to one embodiment of the present invention, the pre-grace period option unit in the reader device 14 provides the best time with an automatic detection, a fixed built-in time length, or a setting based on the user's behavior pattern, and outputs the pre-grace period for the user to confirm the time length thereof. The setting of the pre-grace period is determined based on the user's behavior pattern, which means that the system can automatically determine the pre-grace period by analyzing the user's daily behavior pattern. For example, if the system finds that a user typically performs a specific activity during a specific time period, a longer pre-grace period can determine during that time period, or the pre-grace period can be automatically extended or shorten if the user engaging a high intensity exercise is detected, to ensure the sensor to have sufficient time to complete its pre-grace period.

According to a method for displaying data indicating a glucose level in the biological fluid on a user interface of a reader device according to an embodiment of the present invention, it comprises the following steps (a) and/or (c), or includes steps (a)˜(d):
(a) providing an option unit for the user to set the pre-grace period on the user interface, wherein the pre-grace period is configured to be adjusted to a specific time length in response to a specific implant reaction;
(b) displaying none of the data on the user interface during the pre-grace period;
(c) enabling the user interface to display the data during a data display period in the predetermined usage time length after the pre-grace period expires; and
(d) when the data display period expires, causing the user interface to stop displaying the data.

According to the method mentioned above, the user interface is at least configured to display an option unit for the user to reject or accept setting the specific time length.

According to the method mentioned above, the specific time length is set by one of: (1) selecting from a plurality of preset fixed time lengths; and (2) inputting a user-defined time length by the user.

According to the method mentioned above, the specific time length of the pre-grace period is within a range of half a day to one day.

According to the method mentioned above, the specific time length is not greater than one selected from a group consisting of one day, two days, three days and five days.

According to the method mentioned above, the specific time length is at least 20 minutes.

According to the method mentioned above, a setting of the specific time length is determined based on one or a combination selected from a group consisting of an implant reaction caused by the living body to the sensor, a lifetime of the sensor, the predetermined usage time length, a function of at least one system parameter of the sensor.

According to the method mentioned above, the varying implant reactions from the living bodies are caused by a biological and/or immunological response to the sensor subcutaneously implanted.

According to the method mentioned above, the varying implant reactions include one of a change in a physiological environment in the living body, a sensor electrode passivation, and a cell fibrotic encapsulation of the sensor.

According to the method mentioned above, when the user selects to reject setting the specific time length in the step (a), the one or more processors cause the sensor to start a warm-up period, during which the sensor does not display the data.

According to the method mentioned above, the one or more processors further execute the following step before the step (a): providing the user to set a warm-up period for the sensor, during which the sensor does not display the data.

According to the method mentioned above, when the data display period in the step (c) is close to an end thereof or expires, the one or more processors cause the user interface to remind the user of implanting a second sensor, and when the second sensor has been implanted, the one or more processors cause the user interface to provide a second pre-grace period for the user to set on the user interface.

According to the method mentioned above, when the data display period in the step (c) is close to an end thereof or expires, the user interface displays a post-grace period option unit for setting a post-grace period, and when the post-grace period is set by the user and the data display period expires, the user interface continues to display the data during the post-grace period.

According to the method mentioned above, the signal transmitted from the sensor is a glucose measurement signal, and the data is a glucose reading converted from the glucose measurement signal.

According to the method mentioned above, if the pre-grace period is set by the user in the step (a), the step (a) further comprises causing the user interface to allow the user to confirm the specific time length set for the pre-grace period.

According to the method mentioned above, the step (a) further comprises causing the user interface to display an expiration time message associated with the specific time length for the pre-grace period, and the expiration time message is displayed in a format of one selected from a group consisting of an expiration time point, a countdown timing, a positive timing, and a combination thereof.

According to the method mentioned above, the memory stores each data received by the wireless communication module to form a data set, and during the data display period, the user interface displays the data not greater than 10% of a mean absolute relative difference (MARD) of the data set.

Based on the above, it can be realized that the present invention has the following efficacies:
(1) the present invention provides the option of setting a pre-grace period that allows the user to adapt to the differences in responses of different living bodies to sensor implantation, thereby improving applicability and effectiveness of the subcutaneous glucose monitoring system and providing better monitoring results. The customized pre-grace periods can better reflect the living body's adaptation to the sensor. Accordingly, the system ensures that the monitoring results are more accurate and reliable;
(2) the announced service life of convention sensor is a specific number of days (for example, 15 days). However, it is often difficult to provide accurate measurement readings during 1 hour to 3 days at the beginning after installation. Therefore, the service life of the sensor that the patient/user can actually enjoy is obviously insufficient, resulting in the sacrifice of patient/user's rights. On the contrary, according to the present invention, effective measurement data can be provided throughout the measurement period/data display period, and the actual effective usage life of the product can meet the announced service life, and thus the patient/user's rights are fully protected;
(3) before the service life of the first sensor expires/ends, the patient/user can be reminded to implant the second sensor in time, so that the glucose level monitoring on the patient/user will not be interrupted, and thus any abnormal readings of the patient's glucose and/or alarm resulting therefrom will not be missed; and
(4) the calibration data for the second sensor does not use the data obtained from the first sensor, so the patient/user does not need to perform complicated operations for calibrating the second sensor. These unexpected effects described above are unpredictable or impossible to be achieved by any prior art.

While the invention has been described in terms of what is presently considered to be the most practical and preferred embodiments, it is to be understood that the invention need not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A subcutaneous glucose monitoring system, comprising:
a glucose measurement device including a sensor having a predetermined usage time length, wherein at least a part of the sensor is configured to be implanted subcutaneously in a living body in contact with a biological fluid to output a measured signal or a data indicating a glucose level in the biological fluid; and
a reader device configured to receive and display the data associated with the signal or convert the signal to the data, and provide a user with a pre-grace period for coping with varying implant reactions in different living bodies, wherein the user device comprises:
one or more processors;
a wireless communication module configured to couple with the one or more processors and wirelessly receive the signal or the data from the sensor;
a memory configured to couple with the one or more processors and store an instruction; and
a user interface configured to couple with the one or more processors and be operated by the user to set the pre-grace period and display the data, wherein:
the one or more processors are configured to execute the following steps according to the instruction:
(a) providing an option unit for the user to set the pre-grace period on the user interface, wherein the pre-grace period is configured to be adjusted to a specific time length in response to a specific implant reaction;
(b) displaying none of the data on the user interface during the pre-grace period;
(c) enabling the user interface to display the data during a data display period in the predetermined usage time length after the pre-grace period expires; and
(d) when the data display period expires, causing the user interface to stop displaying the data.

2. The subcutaneous glucose monitoring system according to claim 1, wherein the user interface is at least configured to display the option unit for the user to reject or accept setting the specific time length.

3. The subcutaneous glucose monitoring system according to claim 2, wherein the specific time length is set by one of: (1) selecting from a plurality of preset fixed time lengths; and (2) inputting a user-defined time length by the user.

4. The subcutaneous glucose monitoring system according to claim 1, wherein the specific time length of the pre-grace period is within a range of half a day to one day.

5. The subcutaneous glucose monitoring system according to claim 1, wherein the specific time length is not greater than one selected from a group consisting of one day, two days, three days and five days.

6. The subcutaneous glucose monitoring system according to claim 1, wherein the specific time length is at least 20 minutes.

7. The subcutaneous glucose monitoring system according to claim 1, wherein: a setting of the specific time length is determined based on one or a combination selected from a group consisting of an implant reaction caused by the living body to the sensor, a lifetime of the sensor, the predetermined usage time length, a function of at least one system parameter of the sensor.

8. The subcutaneous glucose monitoring system according to claim 1, wherein the varying implant reactions from the living bodies are caused by a biological and/or immunological response to the sensor subcutaneously implanted.

9. The subcutaneous glucose monitoring system according to claim 8, wherein the varying implant reactions include one of a change in a physiological environment in the living body, a sensor electrode passivation, and a cell fibrotic encapsulation of the sensor.

10. The subcutaneous glucose monitoring system according to claim 1, wherein when the user selects to reject setting the specific time length in the step (a), the one or more processors cause the sensor to start a warm-up period, during which the sensor does not display the data.

11. The subcutaneous glucose monitoring system according to claim 1, wherein the one or more processors further execute the following step before the step (a): providing the user to set a warm-up period for the sensor, during which the sensor does not display the data.

12. The subcutaneous glucose monitoring system according to claim 1, wherein when the data display period in the step (c) is close to an end thereof or expires, the one or more processors cause the user interface to remind the user of implanting a second sensor, and when the second sensor has been implanted, the one or more processors cause the user interface to provide a second pre-grace period for the user to set on the user interface.

13. The subcutaneous glucose monitoring system according to claim 1, wherein when the data display period in the step (c) is close to an end thereof or expires, the user interface displays a post-grace period option unit for setting a post-grace period, and when the post-grace period is set by the user and the data display period expires, the user interface continues to display the data during the post-grace period.

14. The subcutaneous glucose monitoring system according to claim 1, wherein the signal transmitted from the sensor is a glucose measurement signal, and the data is a glucose reading converted from the glucose measurement signal.

15. The subcutaneous glucose monitoring system according to claim 1, wherein if the pre-grace period is set by the user in the step (a), the step (a) further comprises causing the user interface to allow the user to confirm the specific time length set for the pre-grace period.

16. The subcutaneous glucose monitoring system according to claim 15, wherein the step (a) further comprises causing the user interface to display an expiration time message associated with the specific time length for the pre-grace period, and the expiration time message is displayed in a format of one selected from a group consisting of an expiration time point, a countdown timing, a positive timing, and a combination thereof.

17. The subcutaneous glucose monitoring system according to claim 1, wherein the memory stores each data received by the wireless communication module to form a data set, and during the data display period, the user interface displays the data not greater than 10% of a mean absolute relative difference (MARD) of the data set.

18. A subcutaneous glucose monitoring system, comprising:
a glucose measurement device including a sensor having a predetermined usage time length, wherein at least a part of the sensor is configured to be implanted subcutaneously in a living body in contact with a biological fluid to output a measured signal or a data indicating a glucose level in the biological fluid; and
a user device configured to receive and display the data associated with the signal or convert the signal to the data, and provide a user with a pre-grace period for coping with varying implant reactions with different living bodies, wherein the user device comprises:
a processor;
a wireless communication module configured to couple with the processor and wirelessly receive the signal or the data from the sensor;
a memory configured to couple with the processor and store an instruction; and
a user interface configured to couple with the processor and be operated by the user to set the pre-grace period and display the data, wherein:
the processor is configured to provide an option unit for the user to set the pre-grace period on the user interface, wherein the pre-grace period is configured to be adjusted to have a specific time length responding to a specific one of the varying implant reactions according to the instruction.

19. The subcutaneous glucose monitoring system according to claim 18, wherein the processor is further configured to enable the user interface to display the data during a data display period in the predetermined usage time length after the pre-grace period expires.

20. A subcutaneous glucose monitoring system, comprising:
a glucose measurement device including a sensor having a predetermined usage time length by a factory end, wherein at least a part of the sensor is configured to be implanted subcutaneously in a living body in contact with a biological fluid to output a measured signal or a data indicating a glucose level in the biological fluid; and
a reader device configured to receive and display the data associated with the signal or convert the signal to the data, wherein the user device comprises:
a processor;
a wireless communication module configured to couple with the processor and wirelessly receive the signal or the data from the sensor;
a memory configured to couple with the processor and store an instruction; and
a user interface configured to couple with the processor and operated by the user to set the pre-grace period and display the data, wherein:
the processor is configured to provide a user with a pre-grace period for coping with varying implant reactions in different living bodies and enable the user interface to display the data during a data display period in the predetermined usage time length after the pre-grace period expires according to the instruction.
